(19) United States
(12) Patent Application Publication
(10) Pub. No.: US 2012/0220020 A1
(43) Pub. Date: Aug. 30, 2012

(54) MULTICHAMBER PHOTOBIOREACTOR

(75) Inventors: Christoph Mueller-Rees, Pullach (DE); Florian Spruederer, Altoetting (DE); Klaus Wenzeis, Wurmannsquick (DE)

(73) Assignee: WACKER CHEMIE AG, Muenchen (DE)

(21) Appl. No.: 13/501,911

(22) PCT Filed: Oct. 19, 2010
(86) PCT No.: PCT/EP10/65704

§ 371 Date: Apr. 13, 2012

(30) Foreign Application Priority Data
Oct. 20, 2009 (DE) 10 2009 045 853.0

(51) Int. Cl. C12M 3/00; C12N 1/12

(52) U.S. Cl. 435/257.1; 435/292.1

(57) ABSTRACT

A multichamber photobioreactor comprises at least one cultivation chamber and at least one temperature control chamber and is characterized in that at least one outer surface of the cultivation chamber comes in contact with the temperature-controlling medium in such a way that at least 50 percent of at least one outer surface of the cultivation chamber come in contact with the temperature-controlling medium, and in that the components coming in contact with the cultivation medium are made of or coated with silicone materials.

The invention relates to a multichamber photobioreactor with at least one cultivation chamber and at least one attemperation chamber.

Photobioreactors are used for large-scale production of phototrophic organisms, e.g. cyanobacteria or microalgae, for example Spirulina, Chlorella, Chlamydomonas or Haematococcus. These microalgae are able to convert light energy, CO₂ and water into biomass. Photobioreactors of the first generation use sunlight as the light source. The reactors consist of large open tank units of a variety of designs, for example round tank units with diameters up to 45 m and with rotating mixing arms. These reactors are generally made of concrete or plastics. Closed bioreactors are also used in various forms. Closed bioreactors can be plate bioreactors, tubular bioreactors, (bubble) column bioreactors or hose-type bioreactors. To optimize light input, this type of reactor is made of transparent or translucent materials, such as glass or plastic. WO 2008/145719 describes photoreactors that are illuminated with LED plastic moldings, in which LED lamps are embedded in a plastic matrix, preferably a silicone matrix.

In addition to the light input, the temperature of the culture medium is also of great importance for establishing optimum cultivation conditions. Therefore external heat exchangers are often used for temperature control. A plate reactor made of transparent materials such as glass or plastic for the cultivation of phototrophic organisms is described in DE 4134813 A1. A culture medium that is attemperated by means of an external heat exchanger flows through this plate reactor. The object of DE 202005001733 U1 is a solar reactor for plant-like algae and microorganisms, in which the culture medium circulates in a spiral-shaped, transparent tubular reactor. An external heating module is recommended for heating the culture medium. Temperature control of a plate photobioreactor with an external heat exchanger is described in U.S. 2008/0293132, wherein for cooling the plates they can also be equipped with cooling channels. A disadvantage of these embodiments is that temperature control is not continuous throughout the volume of the culture medium. There may therefore be temperature gradients and temperature fluctuations in the culture medium. U.S. 2007/0048848 describes the cultivation of biomasses in troughs, which can be covered with insulating materials. NL 9100715 A describes a tubular reactor for cultivation of algae with a heating element that is separated from the algal medium by insulating material. A disadvantage in using insulating materials is that temperature control is not active.

WO 2009/039317 describes a photobioreactor that is surrounded by a double jacket, in which a gaseous or liquid attemperating medium circulates. The object of WO 98/18903 A1 is a solar element with active or passive temperature control for solar reactors in the form of multiple plates with webs through which liquids can flow, wherein reaction medium or attemperating medium flows through the spaces formed by the webs, so that the surface of the compartment filled with reactor medium comes into contact with the surface of the compartment filled with attemperating medium. U.S. Pat. No. 5,958,761 describes a photobioreactor for cultivation of algae consisting of a cylindrical vessel with an internal, coaxially arranged cylindrical body, each made of glass. The internal cylinder is filled with culture medium, which is surrounded by attemperating medium, which circulates in the outer cylinder. A drawback in these embodiments is the choice of material, which leads to accumulations of microorganisms on the walls of the chamber enclosing the culture medium that can only be removed at great expense.

It should also be noted that cultures of macro- and microorganisms are very sensitive systems, which require conditions that are as constant as possible for successful cultivation. Thus, if the cultivation parameters (light, temperature, flow characteristics) are not constant during the algal growth phase, the quality of the algae is impaired as a result of stress-induced alteration of the metabolic processes. Algae with constant and reproducible properties can only be produced with constant production conditions throughout the entire algal cultivation period. Coating of the surfaces in the cultivation chamber with algae alters these production parameters uncontrollably.

The problem was therefore to construct a closed photobioreactor, which on the one hand ensures uniform and controlled attemperation of the culture medium and on the other hand prevents the accumulation of microorganisms or facilitates the cleaning away of accumulations.

The invention relates to a multichamber photobioreactor with at least one cultivation chamber and at least one attemperation chamber, characterized in that at least one outer surface of the cultivation chamber comes into contact with the attemperating medium, in such a way that at least 50% of at least one outer surface of the cultivation chamber comes into contact with the attemperating medium, and in that the components that come into contact with the culture medium are made of silicone materials or are coated with silicone materials.

The multichamber photobioreactor is suitable for cultivating phototrophic macro- or microorganisms in an aqueous medium. Phototrophic organisms are defined as those that require light and carbon dioxide, or optionally some other carbon source, for growth. Examples of phototrophic macroorganisms are macroalgae, plants, mosses, plant cell cultures. Examples of phototrophic microorganisms are phototrophic bacteria such as purple bacteria and phototrophic microalgae including cyanobacteria. Preferably the multichamber photobioreactor is used for cultivating phototrophic microorganisms, especially preferably for cultivating phototrophic microalgae. Suitable culture media contain, apart from water and macro- or microorganisms, preferably also nutrient salts and/or substances that promote growth or product formation, optionally organic or inorganic carbon sources, for example bicarbonates or sodium hydrogen carbonate. The culture medium can optionally in addition be buffered with respect to the pH.

Water is preferably used as the attemperating medium.

It is important that at least one outer surface of the cultivation chamber comes into contact with the attemperating medium in such a way that the temperature fluctuations in the culture medium are as small as possible. For this, at least 50% of at least one outer surface of the cultivation chamber should come into contact with the attemperating medium. An embodiment in which at least one outer surface of the cultivation chamber comes into contact completely with the attemperating medium is preferred. In embodiments in which an outer surface of the cultivation chamber does not come into contact completely with the attemperating medium, a procedure can also be adopted such that the contact surface is not continuous, but is interrupted—for example by means of constructional elements resembling cooling fins.

The multichamber reactor can be of any design, provided the multichamber principle is adhered to. Hoses, tubes, plates, or bags, in each case of any design, can be used.

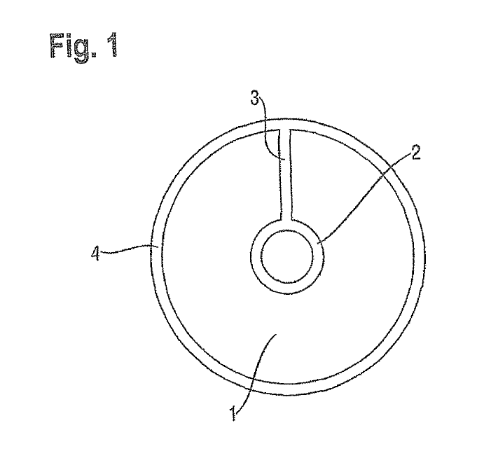

The multichamber photobioreactor with at least one cultivation chamber and at least one attemperation chamber can be in the form of a hose or tube, in each case with round, oval or polygonal cross section. In the following description, the term “hose” also encompasses the embodiment “tube”. For separating the cultivation chamber and the attemperation chamber, the hose can be divided into two or more chambers by installing webs. For example, the hose can be divided into two chambers by means of a radially arranged web. A procedure can also be used in which the hose has one or more internal hoses arranged in its interior, which are optionally joined to the outer hose by a web. In another alternative, one or more hoses with smaller diameter are inserted in an outer hose with larger diameter. A hose that is made up of an outer hose and a coaxially arranged internal hose is preferred. A hose 1 (double hose), which contains a coaxially arranged internal hose 2, which is joined via a web 3 to the outer hose 4, is especially preferred; such as the double hose depicted in FIG. 1.

In the case of the hose-shaped reactor, when it is divided into two chambers, in each case one of the chambers is charged with culture medium and the other chamber with attemperating medium. With more than two chambers, these are preferably charged alternately with culture medium or attemperating medium. In the embodiments with one or more internal hoses in an outer hose, the outer hose can be filled with the culture medium and the internal hose with the attemperating medium. Preferably the internal hose is filled with the culture medium and the outer hose with the attemperating medium.

The multichamber photobioreactor with at least one cultivation chamber and at least one attemperation chamber can also be designed as a plate reactor, wherein two or more plane-parallel plates are firmly joined by means in each case of spaced webs placed between the plates. For lateral closing of the plate reactor, side plates are provided, which are firmly joined to the plane-parallel plates. The resultant chambers of the plate reactor can be charged alternately with culture medium and attemperating medium.

For photobioreactors in the form of bags, two or more bags can be joined together in such a way that in each case a common surface separates the bags. Once again, the bags are preferably charged alternately with culture medium and attemperating medium.

The multichamber photobioreactor is made at least partially, preferably completely, of transparent or translucent materials. Transparent materials are to be understood as those that let through at least 80% of the light in the region of the spectrum from 400 nm to 1000 nm. Translucent materials are to be understood as those that let through at least 50% of the light in the region of the spectrum from 400 nm to 1000 nm. Transparent materials are preferred.

It is important that those regions of the multichamber photobioreactor that are arranged between the culture medium and the light source or light sources for illuminating the culture medium are made of transparent/translucent materials. If the culture medium is in an outer chamber and the attemperating medium in an inner chamber, which are in each case surrounded by the culture medium, the chamber containing the attemperating medium can be made of nontransparent or nontranslucent materials.

Suitable materials are glass and plastics, for example homopolymers or copolymers such as polymethylmethacrylate (Plexiglas), polyesters such as PET, polycarbonate, polyamide, polystyrene, polyethylene, polypropylene, polyvinyl chloride or silicone materials such as silicones or copolymers with silicone and organocopolymer segments.

Silicone materials such as silicones or copolymers with silicone and organocopolymer segments are used for the components of the multichamber photobioreactor that come into contact with the culture medium. A procedure can also be used in which the components of the multichamber photobioreactor that come into contact with the culture medium are coated with silicone materials such as silicones or copolymers with silicone and organocopolymer segments, if they are not made of these materials.

Transparent or translucent silicone materials are especially preferred. Suitable silicone materials are for example addition-crosslinking silicones (silicone rubbers), wherein the addition crosslinking can be initiated thermally or by radiation, and copolymers with silicone and organocopolymer segments (silicone hybrid polymers).

Addition-crosslinking silicone rubber systems contain

a) organosilicon compounds, which have residues with aliphatic carbon-carbon multiple bonds,

b) optionally organosilicon compounds with Si-bonded hydrogen atoms or instead of a) and b)

c) organosilicon compounds, which have residues with aliphatic carbon-carbon multiple bonds and Si-bonded hydrogen atoms, and in each case

d) catalysts promoting the addition of Si-bonded hydrogen onto aliphatic multiple bond and

e) optionally agents that delay the addition of Si-bonded hydrogen onto aliphatic multiple bond at room temperature.

Suitable silicone rubbers crosslinking by addition reaction are high-temperature-vulcanizing (HTV) solid silicone rubbers.

Addition-crosslinking HTV silicone rubbers are obtained by the crosslinking of multiply ethylenically unsaturated groups, preferably vinyl groups, substituted organopolysiloxanes with organopolysiloxanes multiply substituted with Si—H groups in the presence of platinum catalysts.

One of the components of the peroxide- or addition-crosslinking HTV-2 silicone rubbers preferably consists of dialkylpolysiloxanes of structure R₃SiO[—SiR₂O]ₙ—SiR₃ with n≧0, generally with 1 to 4 carbon atoms in the alkyl residue, wherein the alkyl residues can be replaced completely or partially with aryl residues such as the phenyl residue and, at one or at both ends, one of the terminal residues R is replaced with a polymerizable group such as the vinyl group. However, polymers with side or with side and terminal vinyl groups can also be used. Vinyl-end-blocked polydialkylsiloxanes of structure CH₂═CH₂—R₂SiO[—SiR₂O]ₙ—SiR₂—CH₂═CH₂ are preferably used, as well as vinyl-end-blocked polydimethylsiloxanes of the stated structure, which still bear vinyl side groups. In the case of addition-crosslinking HTV silicone rubbers, the second component is a copolymer of dialkylpolysiloxanes and polyalkylhydrogensiloxanes of general formula R′₃SiO[—SiR₂O]ₙ—[SiHRO]ₘ—SiR′₃ with m≧0, n≧0 and with the proviso that at least two SiH groups must be present, wherein R′ can represent H or R. Accordingly there are crosslinking agents with side and terminal SiH groups, whereas siloxanes with R′═H, which only possess terminal SiH groups, are also still used for chain extension. Platinum catalysts are used as crosslinking catalysts. HTV silicone rubbers are also processed as a single-component system.

Other suitable materials are silicone hybrid polymers. Silicone hybrid polymers are copolymers or graft copolymers of organopolymer blocks, for example polyurethane, polyurea or polyvinyl esters, and silicone blocks, generally based on polydialkylsiloxanes of the aforementioned specification. For example, thermoplastic silicone-hybrid polymers are described in EP 1412416 B1 and EP 1489129 B1, the relevant disclosure of which is also to be an object of the present application. Silicone hybrid polymers of this kind are called thermoplastic silicone elastomers (TPSEs). Suitable materials are also (condensation- or radiation-) crosslinkable silicone hybrid materials, as described in WO 2006/058656.

The surface morphology of the silicone hoses is important for inhibition or prevention of fouling with microorganisms. The surface morphology is determined from the contact angle of the surface with water. Surfaces with contact angles between 100° and 120° are preferred, surfaces with contact angles between 100° and 115° are especially preferred, and surfaces with contact angles between 100° and 113° are quite especially preferred. The contact angle is adjusted through selection of the silicone materials. Other measures for increasing the contact angle, for example roughening of the surface (e.g. imitation of the so-called lotus effect), are preferably not used. Such roughening can in fact disturb the cultivation of the phototrophic microorganisms. The contact angle of the surface of the silicone hoses with water can be determined by methods known by a person skilled in the art, for example according to DIN 55660-2, using commercially available measuring instruments for determining the contact angle, for example the contact angle measuring systems obtainable from the company Krüss.

Optionally the stated addition-crosslinked silicones can contain usual additives for promoting adhesion or usual fillers or fibrous materials for improving the mechanical properties. These additives are preferably used at most in amounts such that the silicone molding remains transparent or translucent. Light-conducting additives and light wave-shifting additives can also be added.

Preferably, silicone materials are also used for coating the components that come into contact with the culture medium, especially if the components are not made of the stated silicone materials.

Silicone materials preferred as coating agent are, in addition to the silicone materials already mentioned for production of the components, silicone rubbers crosslinking by condensation even at room temperature, and room-temperature addition-crosslinking silicone rubbers and silicone resins and silicone gels.

Silicone rubbers suitable as coating agents crosslinking at room temperature by condensation are room-temperature-crosslinking 1-component systems, so-called RTV-1 silicone rubbers. The RTV-1 silicone rubbers are organopolysiloxanes with condensable end groups, which crosslink in the presence of catalysts by condensation at room temperature. The commonest are dialkylpolysiloxanes of structure R₃SiO[—SiR₂O]ₙ—SiR₃ with a chain length of n>2. The alkyl residues R can be identical or different and generally have 1 to 4 carbon atoms and can optionally be substituted. The alkyl residues R can also be replaced partially with other residues, preferably with aryl residues, which are optionally substituted, and wherein the alkyl (aryl) groups R are partially exchanged with condensation-crosslinkable groups, for example alcohol residues (alkoxy system), acetate residues (acetic acid system), amine residues (amine system) or oxime residues (oxime system). The crosslinking is catalyzed by suitable catalysts, for example tin or titanium catalysts.

Room-temperature condensation-crosslinking silicone rubbers suitable as coating agent are also room-temperature-crosslinking 2-component systems, so-called RTV-2 silicone rubbers. RTV-2 silicone rubbers are obtained by condensation crosslinking of organopolysiloxanes multiply substituted with hydroxyl groups in the presence of silicic acid esters. Alkylsilanes with alkoxy groups (alkoxy system), oxime groups (oxime system), amine groups (amine system) or acetate groups (acetic acid system) can also be used as crosslinking agents, which in the presence of suitable condensation catalysts, for example tin or titanium catalysts, crosslink with the hydroxyl group-terminated polydialkylsiloxanes.

Examples of the polydialkylsiloxanes contained in RTV-1 and RTV-2 silicone rubber are those of formula (OH)R₂SiO[—SiR₂O]ₙ—SiR₂ (OH) with a chain length of n>2, wherein the alkyl residues R can be identical or different, generally contain 1 to 4 carbon atoms and optionally can be substituted. The alkyl residues R can also be replaced partially with other residues, preferably with aryl residues, which optionally are substituted. Preferably the polydialkylsiloxanes contain terminal OH groups, which crosslink at room temperature with the silicic acid esters or the system alkylsilane/tin(titanium) catalyst.

Examples of the alkylsilanes with hydrolyzable groups, contained in RTV-1 and RTV-2 silicone rubbers, are those of formula RₐSi (OX)₄₋ₐ, with a=1 to 3 (preferably 1), and X with the meaning of R″ (alkoxy system), C(O)R″ (acetic acid system), N═CR″₂ (oxime system) or NR″₂ (amine system), wherein R″ denotes a monovalent hydrocarbon residue with 1 to 6 carbon atoms.

Silicone rubbers suitable as coating agents, and addition-crosslinking at room temperature, are room-temperature-crosslinking 1-component systems, so-called addition-crosslinking RTV-1 silicone rubbers, room-temperature-crosslinking 2-component systems, so-called addition-crosslinking RTV-2 silicone rubbers or also room-temperature-crosslinking multicomponent systems. The crosslinking reaction can be initiated cationically, by means of appropriate catalysts, or radically, by means of peroxides, or by radiation, in particular UV radiation, or thermally.

Addition-crosslinking RTV-2 silicone rubbers are obtained by crosslinking, catalyzed by Pt catalysts, of multiply ethylenically unsaturated groups, preferably vinyl groups, substituted organopolysiloxanes with organopolysiloxanes multiply substituted with Si—H groups in the presence of platinum catalysts.

Preferably one of the components consists of dialkyl polysiloxanes of structure R₃SiO[—SiR₂O]ₙ—SiR₃ with n≧0, generally with 1 to 4 carbon atoms in the alkyl residue, wherein the alkyl residues can be replaced completely or partially with aryl residues such as the phenyl residue, and at one or at both ends one of the terminal residues R is replaced with a polymerizable group such as the vinyl group. Also partially, residues R in the siloxane chain, also in combination with the residues R of the end groups, can be replaced with polymerizable groups. Vinyl-end-blocked polydimethylsiloxanes of structure CH₂═CH₂—R₂SiO[—SiR₂O]ₙ—SiR₂—CH₂═CH₂ are preferably used.

The second component contains an Si—H-functional crosslinking agent. The polyalkylhydrogensiloxanes usually employed are copolymers of dialkylpolysiloxanes and polyalkylhydrogensiloxanes with the general formula R′₃SiO[—SiR₂O]ₙ—[SiHRO]ₘ—SiR′₃ with m≧0, n≧0 and with the proviso that at least two SiH groups must be present, wherein R′ can represent H or R. There are accordingly crosslinking agents with side and terminal SiH groups, whereas siloxanes with R′═H, which only possess terminal SiH groups, can also still be used for chain extension. Small amounts of an organoplatinum compound are contained as crosslinking catalyst.

Moreover, special silicone rubbers have recently become commercially available, which are crosslinked by the addition reaction described, wherein special platinum complexes or platinum inhibitor systems are activated thermally and/or photochemically and thus catalyze the crosslinking reaction.

Silicone resins are also suitable materials for production of the transparent or translucent coating. Generally the silicone resins contain units with the general formula R_{b}(RO)_{c}SiO_{(4-b-c)/2}, in which b is equal to 0, 1, 2 or 3, c is equal to 0, 1, 2 or 3, with the proviso that b+c≦3, and R has the meaning given for it above, which form a highly crosslinked organosilicon network. Silicone resins can be used as solvent-free, solvent-containing or as aqueous systems. Furthermore, it is also possible to use functionalized silicone resins, e.g. silicone resins functionalized with epoxy or amine groups.

Silicone gels are also suitable materials for production of the transparent or translucent coating. Silicone gels are produced from two castable components, which crosslink at room temperature in the presence of a catalyst. One of the components generally consists of dialkylpolysiloxanes of structure R₃SiO[—SiR₂O]ₙ—SiR₃ with n≧0, generally with 1 to 4 carbon atoms in the alkyl residue, wherein the alkyl residues can be replaced completely or partially with aryl residues such as the phenyl residue, and at one or at both ends one of the terminal residues R is replaced with a polymerizable group such as the vinyl group. It is also possible for residues R in the siloxane chain, also in combination with the residues R of the end groups, to be replaced partially with polymerizable groups. Vinyl-end-blocked polydimethylsiloxanes of structure CH₂═CH₂—R₂SiO[—SiR₂O]ₙ—SiR₂—CH₂═CH₂ are preferably used.

The second component contains an Si—H-functional crosslinking agent. The polyalkylhydrogensiloxanes usually employed are copolymers of dialkylpolysiloxanes and polyalkylhydrogensiloxanes with the general formula R′₃SiO[—SiR₂O]ₙ—[SiHRO]ₘ—SiR′₃ with m≧0, n≧0 and with the proviso that at least two SiH groups must be present, wherein R′ can denote H or R. There are accordingly crosslinking agents with side and terminal SiH groups, whereas siloxanes with R′═H, which only possess terminal SiH groups, can still be used for chain extension. Small amounts of an organoplatinum compound are contained as crosslinking catalyst. Mixing the components initiates the crosslinking reaction, and the gel is formed. This crosslinking reaction can be accelerated by the action of heat and/or by electromagnetic radiation, preferably UV radiation.

A detailed review of silicones, their chemistry, formulation and application properties is given for example in Winnacker/Küchler, “Chemical Engineering: Processes and Products, Vol. 5: Organic Intermediates, Polymers”, p. 1095-1213, Wiley-VCH Weinheim (2005).

In a preferred embodiment, the materials for the multichamber photobioreactor can contain usual additives such as fillers or fibrous materials for improving the mechanical properties. These additives are preferably used in maximum amounts such that the material remains transparent or translucent. Light-conducting additives can also be added, or light wave-shifting additives can be added for optimizing the usable radiation yield. Suitable additives are also wavelength-blocking additives, for example for blocking infrared radiation.

The chambers of the multichamber photobioreactor can also have geometric structuring, for example for improving the flow properties or for light scattering. Examples are bumps or indentations in the material of the chambers.

Manufacture can be carried out with the established technologies of plastics processing, which are used for the production of moldings. In particular, in the case of silicones, by extrusion or injection molding for the molding of thermoplastic silicones (thermoplastic injection molding), elastomeric silicones (elastomer injection molding) or thermosetting silicones (thermoset injection molding). Combination processes, e.g. exjection, are also possible.

For coating, the silicones are applied in liquid form, either as pure substance, as solution or in aqueous emulsion. The viscosity of the liquid to be applied for coating is preferably from 10 mPas to 300 000 mPas. Application can be carried out by the usual techniques, preferably brush application, spraying, dipping, knife coating, casting. Dipping and spraying are especially preferred. However, other methods can be used for coating tubes, e.g. sponge application, spin-coating, extrusion or crosshead extrusion, and for flat surfaces it is additionally possible to use application by roll coating, roller coating or the lick-roll process.

The thickness of the coating is generally 10 nm to 1000 μm, preferably 1 μm to 100 μm. Optionally, the reactor parts to be coated can be pretreated to improve the adhesion of the silicones, for example by corona treatment. Optionally the silicones can contain usual additives for promoting adhesion or usual fillers for improving the mechanical properties. These additives are preferably used in maximum amounts such that the silicone coating remains transparent or translucent.

Illumination is generally with sunlight, which can optionally be supplemented with artificial light (artificial light sources). Illuminants containing LEDs are preferably used for artificial illumination. However, other artificial light sources are also suitable, for example fluorescent lamps, neon lamps, metal vapor lamps, inert gas lamps, halogen lamps, sulfur plasma lamps. In the case of illumination with artificial light sources, the cultivation conditions can be optimized by using light sources with defined wavelengths, defined intensity and optionally by means of pulsating light sources. It is also conceivable for the artificial light sources, for example in the form of LED chains, to be installed or incorporated in one or more chambers of the multichamber photobioreactor.

The culture medium containing the phototrophic organisms is generally fed from a storage tank into the corresponding chambers of the multichamber photobioreactor. Feed can be mechanical, by means of a pump. In the multichamber photobioreactor, feed of the culture medium can also take place by means of airlift, i.e. by means of air or by means of an air/CO₂ mixture or also nitrogen as carrier gas, which simultaneously provides supply of CO₂ to the culture medium. However, the supply of CO₂ can also be separate and pulsed, and therefore serve for adjusting the pH in the culture medium.

Separation of the cultivated organisms takes place in a separator, for example by centrifugation, filtration or sedimentation.

The attemperating medium is fed into the corresponding chambers. Feed is preferably pneumatic by pump, in cocurrent or in countercurrent flow to the culture medium. The circuit of the attemperating medium can optionally include a heat exchanger unit for regulating the temperature of the attemperating medium. The temperature of the attemperating medium depends essentially on the ambient temperature and can be adjusted correspondingly.

The operation of the multichamber photobioreactor is preferably organized with automation technology. This includes the automated monitoring and adjustment of specific process parameters such as flow rates, temperature, gas exchange, liquid exchange, density or viscosity, salt content of the culture medium, optionally light in the case of artificial illumination (intensity, wavelength, light/darkness cycle, temporal adjustment/change).

1. A multichamber photobioreactor with at least one cultivation chamber and at least one attemperation chamber, wherein at least one outer surface of the cultivation chamber comes into contact with an attemperating medium, so that at least 50% of at least one outer surface of the cultivation chamber comes into contact with the attemperating medium, and in that components that come into contact with a culture medium are made of silicone materials or are coated with silicone materials, wherein a surface of the silicone materials has a contact angle with water between 100° and 120°.

2. The multichamber photobioreactor as claimed in claim 1, wherein the at least one outer surface of the cultivation chamber comes into contact completely with the attemperating medium.

3. The multichamber photobioreactor as claimed in claim 1 wherein the contact surface is of continuous or intermittent design.

4. The multichamber photobioreactor as claimed in claim 1, which is a hose reactor, tubular reactor, plate reactor or bag reactor.

5. The multichamber photobioreactor as claimed in claim 1, wherein a hose is divided into two or more chambers by installing webs.

6. The multichamber photobioreactor as claimed in claim 1, wherein a hose has one or more internal hoses arranged in an

7. A method of production of phototrophic organisms with a multichamber photobioreactor as claimed in claim 1.

8. The multichamber photobioreactor as claimed in claim 2, wherein the contact surface is of continuous or intermittent design.

9. The multichamber photobioreactor as claimed in claim 8, which is a hose reactor, tubular reactor, plate reactor or bag reactor.

10. The multichamber photobioreactor as claimed in claim 9, wherein a hose is divided into two or more chambers by installing webs.

11. The multichamber photobioreactor as claimed in claim 10, wherein a hose has one or more internal hoses arranged in an interior of the hose.

12. A method of production of phototrophic organisms with a multichamber photobioreactor as claimed in claim 11.
